(19) United States
(12) Patent Application Publication
(10) Pub. No.: US 2006/0045850 A1
(43) Pub. Date: Mar. 2, 2006

(54) Nasal delivery of cyclodextrin complexes of anti-inflammatory steroids

(75) Inventors: Ranga R. Namburi, Plainsboro, NJ (US); Sucharitha Jagini, Edison, NJ (US); Burgise F. Palkhiwala, East Windsor, NJ (US)

Correspondence Address: Richard S. Roberts, P.O. Box 484, Princeton, NJ, 08542-0484, US

(73) Assignee: QPharma, LLC

(21) Appl. No.: 10/930,986

(22) Filed: Aug. 30, 2004

(51) Int. Cl. A61K 31/724; A61K 9/14; A61L 9/04

(52) U.S. Cl. 424/46; 514/58

(57) ABSTRACT

Aqueous, anti-inflammatory steroid compositions in solution form suitable for nasal administration and having a reduced stinging sensation are provided as well as a method for treating inflammation of the nasal mucosa by intranasal administration of anti-inflammatory steroid compositions. These solution compositions may result in enhanced nasal bio-availability. The anti-inflammatory steroid composition suitable for intranasal administration includes an anti-inflammatory steroid in an amount of from about 0.0001% to about 2.0% (w/v); a cyclodextrin in an amount of from about 0.1% to about 20% (w/v); an alcohol co-solvent in an amount of from about 0.2% to about 35% (w/v); a crystallization inhibitor where required, an effective amount of an antimicrobial preservative; an effective amount of an antioxidant; an effective amount of a chelating agent; water; and a pH adjusting agent sufficient to adjust the pH of the composition to from about 4 to about 7.

### BACKGROUND OF THE INVENTION

1. Field of the Invention

The present invention pertains to aqueous solution, anti-inflammatory steroid compositions suitable for nasal administration. The invention also pertains to a method for treating inflammation of the nasal mucosa by intranasal administration of anti-inflammatory steroid compositions. More particularly, the invention pertains to stable anti-inflammatory steroid compositions for intranasal administration having a reduced stinging sensation. The invention formulations in solution form may result in enhanced bioavailability from the nose.

2. Description of the Related Art

Anti-inflammatory steroid compositions suitable for nasal administration are known in the art. Typically these include a cortical steroid such as flunisolide, beclomethasone dipropionate, budenoside, mometasone furoate or fluticasone propionate. Anti-inflammatory steroids are difficult to formulate in aqueous solutions due to their poor solubility in water. Acceptable formulations must be able to dissolve an active compound without precipitation or suspend the active without agglomeration or particle size increase upon storage or undue oxidation of the components, i.e. they must be stable. Suitable formulations must also avoid discomfort to the user. Aqueous compositions of anti-inflammatory steroids such as flunisolide suitable for nasal administration are commercially available, for example under the trademarks Nasalide® and Nasarel®. However, currently available compositions, while safe and effective, are known to cause stinging upon administration in some cases. Such a side effect is particularly undesirable when treating nasal inflammation. Adjuvants such as propylene glycol in higher concentration (more than 10 percent), Polysorbate 80 or Tween 80 suitable for use as solubilizers, however, are often unsuitable for the nasal mucosa and/or have an insufficient solubility. A chronic therapy with such a composition is undesirable.

Nasonex® (Mometasone Furoate), Beconase AQ® (Beclomethasone Dipropionate), Nasacort AQ®A (Triamcinolone Acetonide), Rhinocort® Aqua (Budenoside) and Flonase® (Fluticasone Propionate) are marketed formulations with actives suspended. Absolute bio-availability of these suspension formulations is low and for example the absolute bio-availability of Fluticasone from the suspension formulation when administered nasally is less than 2.0 percent and it has no absorption when administered orally. Hence, there is a clear need for development of clear solution formulations of the steroids. U.S. Pat. No. 6,241,969 provides aqueous compositions containing corticosteroids for nasal and pulmonary delivery which comprises at least 50% by weight of an ethoxylated derivative of vitamin E. U.S. Pat. Nos. 4,782,047 and 4,983,595 show an aqueous steroid formulation for nasal administration, however, no cyclodextrins are taught. U.S. Pat. Nos. 5,089,482, 5,955,454 and WO 00/21503 show aqueous formulations of hormones for nasal administration using cyclodextrins, however, no corticosteroids are shown. U.S. patent application 2004022739 also teaches preparation of nasal spray formulation for use in female contraception and the composition is comprised of a GnRH compound and an estrogenic compound in the form of water soluble complex with a water soluble cyclodextrin. The method used to combine cyclodextrins and hormones in these patents is different from this invention. U.S. Pat. No. 5,089,482 combines cyclodextrin with the hormones through using a solvent and removing the solvent through evaporation process and the complex that is formed in this process is combined with other formulation excipients. Thus there is a need for formation of a clear solution formulation of the steroids using simple and industrially feasible appropriate methods. The compositions of the present invention are stable, preservable, and are suitable for nasal administration of anti-inflammatory steroids and have a reduced stinging tendency. In this invention a minimum amount of co-solvent (adjuvant) is used to dissolve the active steroid with application of heat and it is then combined with aqueous phase containing cyclodextrin. The invention also proves that cyclodextrin forms a complex with the active and remains in solution in spite of the fact that low percentage of cosolvent is used in the formulation. The invention formulations of beclomethasone dipropionate and fluticasone propionate teach the addition of a crystallization inhibitor, such as hydroxy propyl methyl cellulose, in the formulation to prevent precipitation upon storage in view of their extremely low solubility in water and also their molecular size. The solubility and stability of beclomethasone and fluticasone is increased in presence of crystallization inhibitor such as hydroxyl propyl methyl cellulose.

### SUMMARY OF THE INVENTION

The invention provides an anti-inflammatory steroid composition suitable for intranasal administration which comprises:
an anti-inflammatory steroid in an amount of from about 0.0001% to about 2.0% (w/v);
a cyclodextrin in an amount of from about 0.1% to about 20% (w/v);
an alcohol co-solvent in an amount of from about 0.2% to about 35% (w/v);
an effective amount of an antimicrobial preservative;

1an effective amount of an antioxidant;

1an effective amount of a chelating agent;
water; and
a pH adjusting agent sufficient to adjust the pH of the composition to from about 4 to about 7.

The composition may also contain a crystallization inhibitor in an amount of from about 0.01% to 10% W/V.

The invention also provides a method of treating inflammation of the nasal mucosa, which method comprises intranasally administering to a subject in need thereof an anti-inflammatory steroid composition comprising:
an anti-inflammatory steroid in an amount of from about 0.0001% to about 2.0% (w/v);
a cyclodextrin in an amount of from about 0.1% to about 20% (w/v);
an alcohol co-solvent in an amount of from about .2% to about 35% (w/v);
an effective amount of an antimicrobial preservative;
an effective amount of an antioxidant;
an effective amount of a chelating agent;
water; and
a pH adjusting agent sufficient to adjust the pH of the composition to from about 4 to about 7.

The composition may also contain a crystallization inhibitor in an amount of from about 0.01% to 10% W/V.

Commercially available metered dose pumps and bottles are used for filling of the invention formulations.

### DETAILED DESCRIPTION OF THE INVENTION

The composition of the invention includes an anti-inflammatory steroid, such as a corticosteroid. The corticosteroids that are useful in the present invention generally include any steroid produced by the adrenocortex, including glucocorticoids and mineralocorticoids, and synthetic analogs and derivatives of naturally occurring corticosteroids having anti-inflammatory activity. Examples of corticosteroids that can be used in the compositions of the invention include aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, fluticasone propionate, halcinonide, hydrocortisone, icomethasone, meprednisone, methylprednisolone, mometasone paramethasone, mometasone furoate monohydrate, prednisolone, prednisone, tixocortol, triamcinolone, and their respective pharmaceutically acceptable derivatives, such as beclomethasone diproprionate, dexamethasone 21-isonicotinate, icomethasone enbutate, tixocortol 21-pivalate, and triamcinolone acetonide. Particularly preferred are compounds such as beclomethasone diproprionate, budesonide, flunisolide, fluticasone propionate, mometasone and triamcinolone acetonide. In one embodiment, the steroid may be present in the anti-inflammatory steroid composition in an amount of from about 0.0001% to about 2.0% w/v. In another embodiment, the steroid may be present in the anti-inflammatory steroid composition in an amount of from about 0.0005% to about 1.0% w/v. In yet another embodiment, the steroid may be present in the anti-inflammatory steroid composition in an amount of from about 0.001% to about 0.1% w/v.

The composition of the present invention includes a cyclodextrin. Cyclodextrins are a group of structurally related saccharides which are formed by enzymatic cyclization of starch by a group of amylases termed glycosyltransferases. Cyclodextrins are cyclic oligosaccharides, consisting of (α-1,4)-linked α-D-glucopyranose units, with a somewhat lipophilic central cavity and a hydrophilic outer surface. The most common naturally occurring cyclodextrins are α-cyclodextrin, β-cyclodextrin and y-cyclodextrin consisting of 6, 7 and 8 glucopyranose units, respectively. Of these three derivatives, β-cyclodextrin appears to be the most useful pharmaceutical complexing agent due to its cavity size, availability, low cost and other properties. Cyclodextrin derivatives of current pharmaceutical interest include the hydroxypropyl derivatives of α-, β- and γ-cyclodextrin, sulfoalkylether cyclodextrins such as sulfobutylether β-cyclodextrin, alkylated cyclodextrins such as the randomly methylated β-cyclodextrin, and various branched cyclodextrins such as glucosyl- and maltosyl β-cyclodextrin.

In aqueous solutions, cyclodextrins form inclusion complexes with many drugs through a process in which the water molecules located in the central cavity are replaced by either the whole drug molecule, or more frequently, by some lipophilic portion of the drug structure. Once included in the cyclodextrin cavity, the drug molecules may be dissociated through complex dilution, by replacement of the included drug by some other suitable molecule or, the drug may be transferred to the matrix for which it has the highest affinity. Importantly, since no covalent bonds are formed or broken during the drug-cyclodextrin complex formation, the complexes are in dynamic equilibrium with free drug and cyclodextrin molecules. In solution, the complexes are usually prepared by addition of an excess amount of the drug to an aqueous cyclodextrin solution. The suspension formed is and then filtered or centrifuged to form a clear drug-cyclodextrin complex solution.

Useful cyclodextrins for use in the present invention non-exclusively include alkyl cyclodextrins, hydroxy alkyl cyclodextrin, such as hydroxy propyl β-cyclodextrin, carboxy alkyl cyclodextrins and sulfoalkyl ether cyclodextrin, such as sulfo butyl ether β-cyclodextrin. Examples of suitable cyclodextrins for use in the present invention non-exclusively include α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl β-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; 3-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, and combinations thereof. In one embodiment, the cyclodextrin may be present in the anti-inflammatory steroid composition in an amount of from about 0.1% to about 20% w/v. In another embodiment, the cyclodextrin may be present in the anti-inflammatory steroid composition in an amount of from about 1.0% to about 5% w/v. In yet another embodiment, the cyclodextrin may be present in the anti-inflammatory steroid composition in an amount of from about 1.5% to about 2.5 % w/v. A preferred molar ratio of steroid to cyclodextrin ranges from about 1:10 to about 1:800, more preferably from about 1:25 to about 1:200, and most preferably from about 1:50 to about 1:100.

The composition of the present invention includes an alcohol co-solvent, such as propylene glycol, glycofurol, ethoxydiglycol, ethyl alcohol, butyl alcohol, glycerin, hexylene glycol, isopropyl alcohol, polyethylene glycol, polyhydric alcohols, or combinations thereof. Polyhydric alcohols are preferred as co-solvents and propylene glycol is most preferred. In one embodiment, the alcohol may be present in the anti-inflammatory steroid composition in an amount of from about 0.2% to about 35% w/v. In another embodiment, the alcohol may be present in the anti-inflammatory steroid composition in an amount of from about 0.2% to about 10.0% w/v. In still another embodiment the alcohol may be present in the anti-inflammatory steroid composition in an amount of from about 1.0% to about 10.0% w/v. In yet another embodiment, the polyhydric alcohol may be present in the anti-inflammatory steroid composition in an amount of from about 2.0% to about 5.0% w/v.

The composition of the present invention includes an effective amount of an antimicrobial preservative. Preservatives can be used to inhibit microbial growth in the compositions. An “effective amount” of a preservative is that amount necessary to prevent the growth of microorganisms in the composition. The amount of preservative is generally that which is necessary to prevent microbial growth in the composition for a storage period of at least six months. Examples of pharmaceutically acceptable preservatives include benzethonium chloride, butylparaben, methyl paraben, ethyl paraben, propyl paraben, benzalkonium chloride, cetyl pyridinium chloride, thimerosal, chlorobutanol, phenylethyl alcohol, benzyl alcohol, potassium sorbate, sodium benzoate, sorbic acid or combinations thereof. In one embodiment, the antibicrobial preservative may be present in the anti-inflammatory steroid composition in an amount of from about 0.002% to about 0.2% w/v. In another embodiment, the antibicrobial preservative may be present in the anti-inflammatory steroid composition in an amount of from about 0.005% to about 0.1% w/v. In yet another embodiment, the antibicrobial preservative may be present in the anti-inflammatory steroid composition in an amount of from about 0.01% to about 0.05% w/v.

The composition of the present invention includes an effective amount of an antioxidant. The term “antioxidant” refers to a compound or mixture of compounds used in a formulation which is useful for preventing the oxidation of active compound in a composition. An antioxidant must be pharmaceutically acceptable at the concentrations used, and should not interfere with the action of the active compound in the formulation. An “effective amount” of an antioxidant is that amount necessary to prevent undue oxidation of the active compound under normal storage conditions. Presently preferred antioxidants are butylated hydroxyanisole, and to butylated hydroxytoluene. In one embodiment, the antioxidant may be present in the anti-inflammatory steroid composition in an amount of from about 0.0002 to about 0.5% w/v. In another embodiment, the antioxidant may be present in the anti-inflammatory steroid composition in an amount of from about 0.0002% to about 0.05% w/v. In yet another embodiment, the antioxidant may be present in the anti-inflammatory steroid composition in an amount of from about 0.002% to about 0.02% w/v.

The composition of the present invention includes an effective amount of a chelating agent. The term “chelating agent” refers to a compound or mixture of compounds used in a formulation. Chelating agents remove trace amounts of metal ions such as iron, copper and lead and acts as antioxidant synergist as otherwise these heavy metals catalyze oxidation reactions. Presently preferred chelating agents non-exclusively include different salts of edetic acid. These non-exclusively include edetate disodium, edetate calcium disodium, edetate tetrasodium, edetate trisodium, and combinations thereof. In one embodiment, the chelating agent may be present in the anti-inflammatory steroid composition in an amount of from about 0.005% to about 0.1% w/v. In another embodiment, the chelating agent may be present in the anti-inflammatory steroid composition in an amount of from about 0.01% to about 0.05% w/v. In yet another embodiment, the chelating agent may be present in the anti-inflammatory steroid composition in an amount of from about 0.01% to about 0.02% w/v.

In some cases, the composition of the present invention may include a crystallization inhibitor. This is more often preferred with corticosteroids with higher molecular weights such as Fluticasone propionate (500.6) or Beclomethasone dipropionate (539.06). Corticosteroids with molecular weights little lower such as Flunisolide 443.51 and Budenoside 430.5 usually do not require crystallization inhibitor. Presently preferred crystallization inhibitors non-exclusively include hydroxypropyl methyl cellulose, hydroxyethyl cellulose, hydroxypropyl cellulose, methyl cellulose, poly(2-propenoic acid), and other cellulose derivatives, and combinations of these cellulose derivatives with low viscosity grades. Hydroxy propyl methyl cellulose of 6 cps or 3 cps grades may be used in the invention formulations. In one embodiment, the crystallization inhibitor may be present in the anti-inflammatory steroid composition in an amount of from about 0.01% to about 10.0% w/v. In another embodiment, the crystallization inhibitor may be present in the anti-inflammatory steroid composition in an amount of from about 0.1% to about 5.0% w/v. In yet another embodiment, the crystallization inhibitor may be present in the anti-inflammatory steroid composition in an amount of from about 1.0% to about 2.5% w/v.

The composition of the present invention then comprises sufficient water to make-up the anti-inflammatory steroid composition in the desired dosage. Preferably the water is pharmaceutical quality purified water. In one embodiment, the purified water may be present in the anti-inflammatory steroid composition in an amount of from about 85.0% to about 98.0% by volume. In another embodiment, the purified water may be present in the anti-inflammatory steroid composition in an amount of from about 90.0% to about 96% by volume. In yet another embodiment, the purified water may be present in the anti-inflammatory steroid composition in an amount of from about 93.0% to about 95.5% by volume.

The composition of the present invention then comprises an amount of a pH adjusting agent sufficient to adjust the pH of the composition to from about 4 to about 7, preferably from about 4.5 to about 6.5 and more preferably from about 5.0 to about 6.0. Preferred pH adjusting agents non-exclusively include citric acid, acetic acid, fumaric acid, hydrochloric acid, malic acid, nitric acid, phosphoric acid, propionic acid, sulfuric acid, tartaric acid, and combinations thereof.

The clear solution formulations are filled in to commercially available bottles and fit with metered dose pumps for nasal delivery of the drug products. Commercially available metering pumps for nasal route application are used to deliver the appropriate dose of corticosteroid per actuation. Such are available from Valois Pharmaceutical Division, Pfeiffer of America, and Saint-Gobain Calmar, Inc. The delivery dose volumes of metered pumps may vary from about 25 microliters to about 200 microliters.

The following non-limiting examples serve to illustrate the invention.

### EXAMPLE 1

Funisolide Nasal Solution

This example describes the preparation of a nasal solution form of flunisolide in accordance with the methods of the present invention. Ingredients for the preparation of a flunisolide nasal solution of the invention are set forth in the table below.

| Ingredient | % quantity | per 200 mL | |
|---|---|---|---|
| Flunisolide | 0.025 | 0.05 | g |
| Hydroxypropyl β Cyclodextrin | 1.5 | 3.0 | g |
| Citric Acid, Anhydrous | 0.002 | 0.004 | g |
| Edetate Disodium | 0.02 | 0.04 | g |
| Propylene glycol | 2.5 | 5.0 | g |
| Butylated hydroxy anisole | 0.002 | 0.004 | g |
| Cetyl pyridium chloride | 0.05 | 0.1 | g |
| Purified water | | QS to 200 | mL |

Process: Propylene glycol is placed in a glass beaker and the contents of the beaker maintained in a hot water bath at a temperature of 50-55° C. Add and dissolve flunisolide under stirring. Continue stirring until it forms a clear solution. Cool the solution to ambient temperature and add and dissolve butylated hydroxy anisole. Separately dissolve hydroxypropyl beta cyclodextrin in purified water 160 mL. To this add the flunisolide dissolved in propylene glycol under stirring. Make solutions of edetate disodium in purified water 10 mL, cetyl pyridinium chloride in purified water 10 mL and citric acid in purified water 10 mL and add each ingredient under stirring to the main bulk. Make up the volume with purified water to the batch size. Check and adjust the pH of the solution and filter the solution through a 0.45 micron nylon membrane filter.

### EXAMPLE 2

Beclomethasone Dipropionate Nasal Solution

This example describes the preparation of a nasal solution form of beclomethasone dipropionate in accordance with the methods of the present invention. Ingredients for the preparation of beclomethasone dipropionate nasal solution of the invention are set forth in the table below.

| Ingredient | % quantity | per 200 mL | |
|---|---|---|---|
| Beclomethasone dipropionate | 0.020 | 0.04 | g |
| Hydroxypropyl β-cyclodextrin | 2.0 | 4.0 | g |
| Citric acid, anhydrous | 0.002 | 0.004 | g |
| Edetate disodium | 0.01 | 0.02 | g |
| Propylene glycol | 5.0 | 10.0 | g |
| Hydroxypropyl methyl cellulose | 2.0 | 4.0 | g |
| 6 cps grade | | | |
| Potassium sorbate | 0.01 | 0.02 | g |
| Purified water | | QS to 200 | mL |

Process: Place propylene glycol in a glass beaker and place the contents of the beaker in a hot water bath maintained at a temperature of 60° C.-70° C. and add and dissolve beclomethasone under stirring. Continue stirring until it forms a clear solution. Separately dissolve hydroxypropyl beta cyclodextrin in purified water 160 mL. To the cyclodextrin solution, add and dissolve under stirring hydroxypropyl methyl cellulose and stir until it forms a clear solution. To this add beclomethasone dissolved in propylene glycol under stirring. Make solutions of edetate disodium in purified water 10 mL, potassium sorbate in purified water 10 mL and citric acid in purified water 10 mL and add each ingredient under stirring to the main bulk. Make up the volume to the batch size with purified water. Check and adjust the pH of the solution and filter the solution through 0.45 micron nylon membrane filter.

### EXAMPLE 3

Fluticasone Propionate Nasal Solution

This example describes the preparation of a nasal solution form of fluticasone propionate in accordance with the methods of the present invention. Ingredients for the preparation of fluticasone propionate nasal solution of the invention are set forth in the table below.

| Ingredient | % quantity | per 200 mL | |
|---|---|---|---|
| Fluticasone propionate | 0.005 | 0.010 | g |
| Sulfobutyl ether β cyclodextrin | 2.0 | 4.0 | g |
| Citric acid, anhydrous | 0.002 | 0.004 | g |
| Edetate disodium | 0.02 | 0.04 | g |
| Propylene glycol | 2.5 | 5.0 | g |
| Hydroxypropyl methyl cellulose | 2.0 | 4.0 | g |
| 6 cps grade | | | |
| Potassium sorbate | 0.01 | 0.02 | g |
| Purified water | | QS to 200 | mL |

Process: Place propylene glycol 5.0 g and water 0.5 g mixture in a glass beaker and place the contents of the beaker in a hot water bath maintained at a temperature of 60° C.-80° C. and add and dissolve fluticasone propionate under stirring. Continue stirring until it forms a clear solution. Separately dissolve sulfobutyl ether beta cyclodextrin in purified water 160 mL. To this cyclodextrin solution, add and dissolve under stirring hydroxypropyl methyl cellulose and stir until it forms a clear solution. To this add fluticasone dissolved in propylene glycol under stirring. Make solutions of edetate disodium in purified water 10 mL, potassium sorbate in purified water 10 mL and citric acid in purified water 10 mL and add each ingredient under stirring to the main bulk. Make up the volume to the batch size with purified water. Check and adjust the pH of the solution and filter the solution through 0.45 micron nylon membrane filter.

### EXAMPLE 4

Budenoside Nasal Solution

This example describes the preparation of a nasal solution form of Budenoside in accordance with the methods of the present invention. Ingredients for the preparation of budenoside nasal solution of the invention are set forth in the table below.

| Ingredient | % quantity | per 200 mL | |
|---|---|---|---|
| Budenoside | 0.025 | 0.05 | g |
| Hydroxypropyl β cyclodextrin | 2.0 | 4.0 | g |
| Citric acid | 0.002 | 0.004 | g |
| Edetate disodium | 0.01 | 0.02 | g |
| Propylene glycol | 2.5 | 5.0 | g |
| Potassium sorbate | 0.01 | 0.02 | g |
| Purified water | | QS to 200 | mL |

Process: Place propylene glycol in a glass beaker and place the contents of the beaker in a hot water bath maintained at a temperature of 60° C.-70° C, and add and dissolve flunisolide under stirring. Continue stirring until it forms a clear solution. Cool the solution to ambient temperature.

Separately dissolve hydroxypropyl beta cyclodextrin in purified water 160 mL. To this add budenoside dissolved in propylene glycol under stirring. Make solutions of edetate disodium in purified water 10 mL, potassium sorbate in purified water 10 mL and citric acid in purified water 10 mL and add each ingredient under stirring to the main bulk. Make up the volume with purified water to the batch size. Check and adjust the pH of the solution and filter the solution through 0.45 micron nylon membrane filter.

### EXAMPLE 5

Nasal Acceptability

The following example illustrates nasal acceptability of the compositions of Examples 1-4.

A series of volunteers are randomly divided into four groups. Group 1 receives the composition of Example 1, Group 2 receives the composition of Example 2, Group 3 receives the composition of Example 3 and Group 4 receives the composition of Example 4. The tests are performed by applying one spray of the compositions to each nostril. Immediately after administration, no noticeable nasal stinging is noticed with the invention formulations. The results indicate superior nasal acceptability for the nasally delivered drug compositions.

### EXAMPLE 6

Acceptable Recoveries:

Using liquid chromatographic run conditions, contents of steroids in solution form are tested. The solutions are passed through 0.45 micron acrodisk glass membrane filter to remove any insoluble actives.

More than 98 percent assay values of the four examples indicate that the active steroids are in true solution form. Even when stored at 8-15° C. the samples show assay values above 98 percent of the quantities added.

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

What is claimed is:

1. An anti-inflammatory steroid composition suitable for intranasal administration which comprises:
an anti-inflammatory steroid in an amount of from about 0.0001% to about 2.0% (w/v);
a cyclodextrin in an amount of from about 0.1% to about 20% (w/v);
an alcohol co-solvent in an amount of from about 0.2% to about 35% (w/v);
an effective amount of an antimicrobial preservative;
an effective amount of an antioxidant;
an effective amount of a chelating agent;
water; and
a pH adjusting agent sufficient to adjust the pH of the composition to from about 5 to about 7.

2. The composition of claim 1 wherein the alcohol co-solvent is present in an amount of from about 0.2% to about 10% (w/v);

3. The composition of claim 1 wherein the anti-inflammatory steroid comprises aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, fluticasone propionate, halcinonide, hydrocortisone, icomethasone, meprednisone, methylprednisolone, mometasone paramethasone, mometasone furoate monohydrate, prednisolone, prednisone, tixocortol, triamcinolone, beclomethasone diproprionate, dexamethasone 21-isonicotinate, fluticasone propionate, icomethasone enbutate, tixocortol 21-pivalate, and triamcinolone acetonide, or combinations thereof.

4. The composition of claim 1 wherein the anti-inflammatory steroid comprises flunisolide, beclomethasone dipropionate, budenoside, fluticasone propionate, mometasone furoate or combinations thereof.

5. The composition of claim 1 wherein the cyclodextrin comprises α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl β-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; 3-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, and combinations thereof.

6. The composition of claim 1 wherein the cyclodextrin comprises hydroxypropyl β-cyclodextrin, sulfobutyl ether β-cyclodextrin, or combinations thereof.

7. The composition of claim 1 wherein the alcohol co-solvent comprises propylene glycol, glycofurol, ethoxydiglycol, ethyl alcohol, butyl alcohol, glycerin, hexylene glycol, isopropyl alcohol, polyethylene glycol, polyhydric alcohols, or combinations thereof.

8. The composition of claim 1 wherein the antibicrobial preservative comprises benzethonium chloride, butylparaben, methyl paraben, ethyl paraben, propyl paraben, benzalkonium chloride, cetyl pyridinium chloride, thimerosal, chlorobutanol, phenylethyl alcohol, benzyl alcohol, potassium sorbate, sodium benzoate, sorbic acid or combinations thereof.

9. The composition of claim 1 wherein the chelating agent comprises a salt of editic acid, or combinations thereof.

10. The composition of claim 1 wherein the pH adjusting agent comprises citric acid, acetic acid, fumaric acid, hydrochloric acid, malic acid, nitric acid, phosphoric acid, propionic acid, sulfuric acid, tartaric acid, or combinations thereof.

11. The composition of claim 1 which comprises flunisolide, hydroxypropyl β-cyclodexrin, citric acid, edetate disodium, propylene glycol, butylated hydroxy anisole, and cetyl pyridium chloride.

12. The composition of claim 1 which comprises beclomethasone dipropionate, hydroxypropyl β-cyclodextrin, anhydrous citric acid, edetate disodium, propylene glycol, hydroxy propyl methyl cellulose and potassium sorbate.

13. The composition of claim 1 which comprises budenoside, hydroxypropyl β-cyclodexrin, citric acid, edetate disodium, propylene glycol, and potassium sorbate.

14. The composition of claim 1 which comprises fluticasone propionate, sulfobutyl ether β-cyclodextrin, anhydrous citric acid, edetate disodium, propylene glycol, hydroxy propyl methyl cellulose and potassium sorbate.

15. The composition of claim 1 which further comprises a crystallization inhibitor.

16. The composition of claim 1 which further comprises a crystallization inhibitor in an amount of 0.5% to about 5.0% w/v.

17. The composition of claim 1 which further comprises a crystallization inhibitor selected from the group consisting of hydroxypropyl methyl cellulose, hydroxyethyl cellulose, hydroxypropyl cellulose, methyl cellulose, poly(2-propenoic acid), and combinations thereof.

18. A method of treating inflammation of the nasal mucosa, which method comprises intranasally administering to a subject in need thereof an anti-inflammatory steroid composition comprising:
an anti-inflammatory steroid in an amount of from about 0.0001% to about 2.0% (w/v); a cyclodextrin in an amount of from about 0.1% to about 20% (w/v);
an alcohol in an amount of from about .2% to about 35% (w/v);
an effective amount of an antimicrobial preservative;
an effective amount of an antioxidant;
an effective amount of a chelating agent;
water; and
a pH adjusting agent sufficient to adjust the pH of the composition to from about 5 to about 7.

19. The method of claim 18 wherein the anti-inflammatory steroid composition comprises flunisolide, hydroxypropyl cyclodexrin, citric acid, edetate disodium, propylene glycol, butylated hydroxy anisole, and cetyl pyridium chloride.

20. The method of claim 18 wherein the anti-inflammatory steroid composition comprises beclomethasone dipropionate, hydroxypropyl cyclodextrin, anhydrous citric acid, edetate disodium, propylene glycol, hydroxy propyl methyl cellulose and potassium sorbate.

21. The method of claim 18 wherein the anti-inflammatory steroid composition comprises budenoside, hydroxypropyl cyclodexrin, citric acid, edetate disodium, propylene glycol, and potassium sorbate.

22. The method of claim 18 wherein the anti-inflammatory steroid composition comprises fluticasone propionate, sulfobutyl ether beta cyclodextrin, anhydrous citric acid, edetate disodium, propylene glycol, hydroxy propyl methyl cellulose and potassium sorbate.
